(19) United States
(12) Patent Application Publication
(10) Pub. No.: US 2011/0196036 A1
(43) Pub. Date: Aug. 11, 2011

(54) USE OF BETAINE

(75) Inventor: Kirsti Jutila, Espoo (FI)

(73) Assignee: DANISCO A/S, Copenhagen K (DK)

(21) Appl. No.: 13/122,853

(22) PCT Filed: Oct. 15, 2009
(86) PCT No.: PCT/FI09/50830

§ 371 Date: Apr. 6, 2011

(30) Foreign Application Priority Data
Oct. 15, 2008 (FI) 20085971

(51) Int. Cl. A61K 31/205; A61P 17/00

(52) U.S. Cl. 514/556

(57) ABSTRACT

The present invention relates to use of betaine for protecting skin from biological irritation. The invention also relates to the use of betaine as a protecting agent in a cosmetic and/or toiletry preparation. The invention further relates to a method of protecting skin from biological irritation.

### FIELD OF THE INVENTION

The present invention relates to use of betaine for protecting skin from biological irritation. The invention also relates to the use of betaine as protecting agent in a cosmetic and/or toiletry preparation. The invention further relates to a method of protecting skin from biological irritation.

### BACKGROUND OF THE INVENTION

Skin is prone to several types of irritation, such as mechanical, thermal, chemical and/or biological irritation.

Patent publication EP 531 387 B discloses that betaine is effective in neutralizing the skin-irritating properties of a cosmetic composition. Publication EP 930 870 B describes that betaine protects skin from mechanical irritation, such as scratching, abrading and cutting.

In addition to these publications, Rigano, L., et al. (2000) Cosmetics & Toiletries magazine Vol. 115, No.12, p. 47-54 describe generally the benefits of betaine in personal care formulations in the cosmetic's field.

Patent publication U.S. Pat. No. 6,660,304 describes a method for treating Miliaria rubra, commonly known as prickly heat, by topically applying a composition comprising betaine and Scutellaria baicalensis extract. Miliaria rubra is a skin disease or disorder, clinical reaction consisting of red, itchy or stingingly blistering pimples. Typically, the lesions are found in the skin of the body area.

It does not appear from the above-mentioned publications that betaine protects skin from biological irritation.

### BRIEF DESCRIPTION OF THE INVENTION

The present invention relates to use of betaine for protecting skin from biological irritation. Further, the present invention relates to the use of betaine for protecting scalp from biological irritation.

The present invention relates also to method of protecting skin from biological irritation comprising applying an effective amount of betaine to the skin. Further, the present invention relates to the method of protecting scalp from biological irritation comprising applying an effective amount of betaine to the scalp.

### BRIEF DESCRIPTION OF THE DRAWINGS

FIG. 1 shows the results with regard to scalp itchiness received in Example 2.

FIG. 2 shows the results with regard to change of general condition received in Example 3.

### DETAILED DESCRIPTION OF THE INVENTION

Skin is continuously subjected to various conditions that expose it to biological irritation. According to the present invention biological irritation is induced by a compound secreted by the mammalian body itself, such as sweat or sebum, or an ingredient or a mixture of ingredients thereof.

It has now been discovered that betaine protects skin from biological irritation.

In the present invention betaine refers to trimethylglycine. The compound is also called trimethylammonioacetate, 1-carboxy-N,N,N-trimethylmethaneaminium, inner salt and glycine betaine. It is a naturally occuring quaternary ammonium type compound having the formula

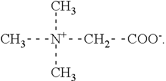

Betaine has a bipolar structure comprising a hydrophilic moiety (COO⁻) and a hydrophobic moiety (N⁺) capable of neutralizing both acid and alkaline solutions.

In its pure form, betaine is a white crystalline compound that is readily soluble in water and lower alcohols. In the present invention betaine can be used, for example, as anhydrous form, or as a monohydrate or a salt. Betaine is commercially available from Finnfeeds Finland Oy as an anhydrous form and also as a monohydrate.

In the present invention, biological irritation refers to the irritation induced by a compound secreted by the mammalian body through the skin. Secretory products, such as sweat and sebum, or an individual ingredient or a mixture of ingredients included in these secretory products may irritate the skin of the body and the scalp especially. Biological irritation is typically temporary by nature. Further, biological irritation is often induced by the environment and/or the circumstances, such as the physical effort and/or exercise. Thus, it is usually also self-inflicted by nature. According to the present invention, biological irritation is not a skin disease or a disease affecting sebaceous glands and/or sweat glands.

In the present invention protecting refers to protecting, preventing and/or alleviating the biological irritation and the symptoms of the irritation, such as itching, stinging and/or burning. The aforementioned symptoms, itching, stinging and burning, can also be symptoms of dry skin. However, the irritation of skin due to dehydration or drying of skin, as such, is not biological irritation according to the present invention.

Skin and scalp are continuously subjected to various conditions that expose it to biological irritation, for example to sweat. In some professions and hobbies the circumstances of the work or the activity expose people to sweating, for example. In addition, there are several professions and also hobbies that require use of protecting equipments, such as, a helmet, a scarf or a cap that by sheltering and covering the skin prevent and/or complicate the evaporation of the secretory products such as the sweat or the sebum, or the individual ingredients of the these secretory products from the skin and/or scalp surface and thus multiply the effect of the biological irritation. Thus, for example the regular use of a helmet, a scarf and/or a cap multiplies the irritating effect of sweat and/or sebum on the scalp. The same applies for the use other protecting equipments and the areas of skin they cover. An area of skin, not necessarily covered by protecting equipment, but otherwise prone to biological irritation, is armpit (axillary fossa). The irritation may be sensed as itching, stinging and/or burning, for example.

Often, for example a scarf or a cap is used under the helmet, which together with hair potentiates the effect of the biological irritation of the scalp.

In addition to sweat, skin generally and scalp specifically excretes sebum. For example exercise, heat, humidity and hormonal action are known to increase the excretion of sebum as well as sweat. Excretion of sebum increases also when scalp is massaged or rubbed and/or hair is brushed.

Sweat contains in addition to water and sodium chloride small amounts of various compounds, such as, potassium chloride, phosphates, amino acids, urea, lactic acid and cresols. Several types of biological waste material and/or excreta are also removed from the body through sweating and excretion of sebum. Sweating and also the secretion of sebum belong to the means of excretion of a mammalian body.

According to the present invention, betaine could be applied to skin before and/or after the occasion or event skin is supposed to be exposed to biological irritation. Naturally, the favorable effects of betaine in protecting the skin from biological irritation are achieved when betaine is applied to the skin on continuous basis.

In the present invention a cosmetic or toiletry preparation refers to a skin and/or hair care or a skin and/or hair cleaning/washing product. In general betaine could be applied to the skin in the form of soap, or a conventional emulsion, lotion or cream composition for example. Specifically, when scalp is in question, betaine could be applied to the scalp also in the form of a hair care product, such as but not limited to, an aqueous solution or a shampooing, conditioning or styling formulation. A hair care composition may be in the form of a lotion, mousse, spray, gel, wax or mask. It may be a rinse-off or a leave-on formulation. The cosmetic or toiletry preparations of the invention can be formulated by common methods well known to those skilled in the art.

In one embodiment of the invention betaine is applied to the skin, in general, in a formulation of a skin-care product that is not rinsed from the skin after its application and specifically to the scalp in the form of a leave-in formulation of a hair care product. Leave-on formulation referes herein to a formulation of a skin-care product that is not rinsed from the skin after its application and/or to a leave-on formulation of a hair care product.

Betaine can be included in a preparation in an amount of 0.1 to 50% by weight of the preparation. A cosmetic or toiletry preparation protecting skin from biological irritation according to the present invention contains betaine in an amount of 0.1 to 20% by weight. In one embodiment of the invention, the cosmetic or toiletry preparation contains betaine 0.2 to 10%, preferably 0.5 to 7% by weight and in another embodiment of the invention, the cosmetic or toiletry preparation contains betaine 0.75 to 5% by weight based on the total weight of the preparation. In one embodiment of the invention, betaine is the sole ingredient protecting skin from biological irritation in the cosmetic and/or toiletry preparation. The preparation may also contain one or several ingredients and additives that are commonly used in cosmetic or toiletry preparations. These include, for example, skin care agents, softening agents, astringent agents, refreshing agents, emulsifying agents, moisturizing agents, stabilizing agents, viscosity regulating agents, emollients, thickeners, surfactants, colouring agents, perfumes and the like, as well as alcohols and/or water. The preparations can be formulated by methods known to those skilled in the art.

The following examples illustrate the present invention. The examples are not to be construed to limit the claims in any manner whatsoever.

### EXAMPLE 1

A shampoo and a leave-on conditioner formulations incorporating betaine in the form of monohydrate were developed. During the development care was taken to restrict the extent of other components in the formulation, so that the properties beneficial with regard to protection of biological irritation can be attributed to the use of betaine, rather that to a plethora of other materials.

Formulation of a shampoo containing 5% (w/w) betaine:

| Trade Name and Supplier | INCI Names | % w/w |
|---|---|---|
| Deionised water | Aqua | to 100 |
| Surfac LCX (Surfachem) | Sodium Lauryl Sulphate | 14.0 |
| | Methyl Chloro Isothiazolinone | ~ |
| | Methyl Isothiazolinone | ~ |
| | Magnesium Chloride | ~ |
| | Magnesium Nitrate | ~ |
| Betafin BP20 (Danisco) | Betaine | 5.0 |
| Cutina AGS (Univar) | Glycol Distearate | 2.0 |
| Laurex CS (S Black) | Cetyl Alcohol | 1.0 |
| Cocamide MEA (S Black) | Cocamide MEA | 1.0 |
| EDTA (S Black) | Tetrasodium EDTA | 0.3 |
| DMDMH (S Black) | DMDM Hydantoin | 0.3 |
| N-Hance 3205 (Crestchem) | Guar hydroxypropyltrimonium | 0.2 |
| | chloride | |
| Methyl Paraben (Clariant) | Methyl Paraben | 0.1 |
| Sodium Chloride (Merck) | Sodium Chloride | ~ |
| Sodium Hydroxide (Merck) | Sodium Hydroxide | ~ |
| | Fragrance | 0.2 |

~ refers to an amount below 0.1% w/w

Formulation of a leave-on conditioner containing 2% (w/w) betaine:

| INCI Names | % w/w |
|---|---|
| Aqua | 92.45 |
| Betaine | 2.0 |
| PEG/PPG-20/15 Dimethicone | 2.0 |
| Amodimethicone | 1.75 |
| Cetrimonium Chloride | 0.68 |
| Trideceth-12 | 0.55 |
| PEG-40 Hydrogenated Castor Oil | 0.25 |
| Fragrance | 0.2 |
| Disodium EDTA | 0.1 |
| Magnesium Nitrate | ~ |
| Alpha-isomethyl Ionone | ~ |
| Benzyl Salicylate | ~ |
| Methylchloroisothiazolinone | ~ |
| Linalool | ~ |
| Geraniol | ~ |
| Magnesium Chloride | ~ |
| Citronellol | ~ |
| Methylisothiazolinone | ~ |

~ refers to an amount below 0.1% w/w

In the first trial, a panel of female ringette players tested the shampoo containing 5% (w/w) betaine (the formulation specified above) and found it to alleviate itching and stinging and protect the scalp from irritation.

In the second trial, the panelists tested the leave-on conditioner containing 2% (w/w) betaine (the formulation specified above) and compared it with a leave-on conditioner without betaine. In this trial, all the panelists used same commercially available shampoo. The results received in the second trial are described in Examples 2 and 3.

### EXAMPLE 2

The effect of betaine on biological irritation was studied with 17 female ringette players wearing a helmet for 1 to 1,5 hours four times a week. The panelists applied two types of leave-on hair conditioner, one set of products containing betaine and one set of products without betaine. 9 panellists used the betaine products for three weeks and then the products without betaine for three next weeks. For the other 8 panellists the order was reversed, first without betaine, then with betaine. The conditioner with betaine contained it at a level of 2% w/w.

The panellists rated their scalp and hair condition after each week. The condition was rated along several dimensions, e.g. scalp itchiness, hair greasiness, ease of breakage, and general condition. The rating scale was a 9-level subjective scale, number 1 referring to a very poor condition and number 9 referring to a very good condition.

The research questions analyzed were whether betaine affects more favourably the hair condition than the other product. First, each hair condition dimension was compared week by week, betaine vs. non-betaine, using the paired-sample t-test (so there were at most 3·17=51 pairs to compare). The scalp itchiness was found to be significantly lower when betaine products were used (p=0.013, n=51).

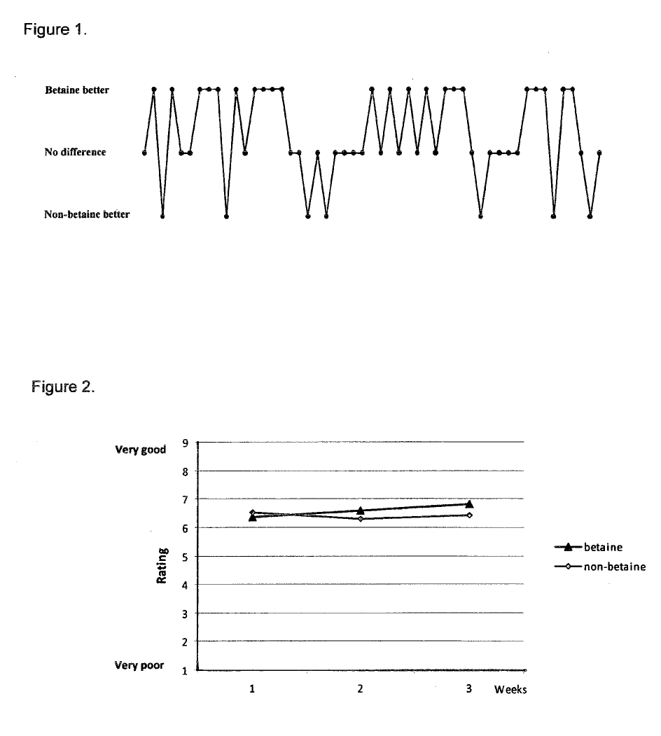

Because the panellists used the scale possibly differently, the results were transformed into a simple dichotomic scale, in order to answer the question “are the betaine products better or worse than the products without betaine”. Applying the binomial test it was found that there is a strong statistical difference in scalp itchiness in favour of betaine (p=0.005, n=29). Also, the ease of breakage was lower when betaine products were used (p=0.044, n=28). FIG. 1 shows the results with regard to scalp itchiness.

### EXAMPLE 3

Another view to compare these two types of leave-on hair conditioner, one set of products containing betaine and one set of products without betaine, is to ask “do the products improve the hair condition when they are used for some time”. Therefore, a linear regression analysis was applied to each three-week period. The slopes (how much the hair condition has improved or deteriorated) for betaine products and non-betaine products were then compared. The general condition was in favour of betaine products (p=0.058, n=17, paired-sample t-test). The average result of all panelists is shown in FIG. 2.

1-16. (canceled)

17. A method of protecting skin from irritation induced by a compound secreted by the mammalian body through the skin comprising applying an effective amount of betaine to the skin.

18. The method according to claim 17, wherein the compound is a secretory product and/or an individual ingredient thereof.

19. The method of according to claim 17 or 18, wherein the skin is scalp.

20. The method of according to claim 17 wherein the irritation is itching, stinging and/or burning of the skin.

21. The method according to claim 17, wherein betaine is applied to the skin in a skin or hair care composition.

22. The method of claim 21, wherein the skin or hair care composition is a cosmetic or toiletry composition.

23. The method of claim 22, wherein the cosmetic or toiletry composition comprises one of more cosmetically acceptable ingredients and betaine.

24. The method according to claim 21, wherein the composition comprises betaine 0.2 to 10% .

25. The method according to claim 21, wherein the composition comprises betaine 0.5 to 7% by weight, based on the total weight of the composition.

26. The method according to claim 21, wherein the composition comprises betaine 0.75 to 5% by weight, based on the total weight of the composition.

27. The method according to any one of claims 21 to 26 wherein the composition is formulated into a leave-on formulation.
